(19) United States
(12) Patent Application Publication
(10) Pub. No.: US 2007/0199238 A1
(43) Pub. Date: Aug. 30, 2007

(54) Method for the production of fatty acid alkyl ester

(76) Inventor: Jeffrey D. Hooker, Birmingham, AL (US)

Correspondence Address: LANIER FORD SHAVER & PAYNE P.C., P O BOX 2087, HUNTSVILLE, AL, 35804, US

(21) Appl. No.: 11/788,674

(22) Filed: Apr. 20, 2007

Related U.S. Application Data
(62) Division of application No. 10/630,097, filed on Jul. 29, 2003.

(51) Int. Cl. C10L 1/32; C07C 51/00

(52) U.S. Cl. 44/301; 554/169

(57) ABSTRACT

An apparatus and method for producing fatty acid alkyl esters from fatty acids derived from vegetable oils and animal fats with an alkaline solution dissolved in stoichiometric or near-stoichiometric levels of a monoalkyl alcohol to form a mixture. The method further comprises emulsifying the mixture as a means to reach a completed chemical reaction state in a reactor section, wherein the oils or fats are transesterified into fatty acid alkyl esters. The transesterification occurs when the natural boundary surfaces of the immiscible mixture are enlarged by ultrasonic cavitation in the reaction section and the transesterification is performed at, or near atmospheric pressure. The method finally includes, after reaching the chemical reaction state, separating residues from the fatty acid alkyl ester in a gravitational phase separation section.

### BACKGROUND OF THE INVENTION

1. Field of the Invention

This invention generally relates to a method and apparatus for producing fatty acid alkyl ester. In particular, the present invention relates to a method and apparatus for the production of fuel for diesel combustion engines. More particularly, the present invention relates to a method and apparatus for the production of fuel capable of meeting specifications without requiring additional treatment.

2. Discussion of Background

The transesterification of vegetable oils or animal fats with a monoalkyl alcohol to produce fatty acid alkyl esters and glycerol is a three step process. First the normally immiscible oil and alcohol phases are brought into intimate contact in order for the ester bond between the fatty acids in the triglyceride to be exchanged for an ester link between the fatty acid and the monoalkyl portion of the alcohol, while the hydroxyl of the alcohol is added to the glyceride backbone of the triglyceride. This exchange is solubility limited, so combinations of solubilizing catalysts, co-solvents, and intense mixing are used to promote solubilization of the alcohol in the oil phase.

During the second step of the reaction, the reaction mixture is a single phase, and the rate is limited by temperature and concentration of the reactants. In the third step of the reaction, the rate is slowed by the presence of the product glycerol. The glycerol is immiscible with the product ester phase, absorbs excess alcohol, and absorbs both acid and alkali catalysts.

Such methods are known whereby the alkaline solution compounded in alcohol is mixed in a mixing tank with the fat or oil for between about 20 minutes and for as much as 1 hour. After mixing, the mixture is left to stand. This sedimentation process takes approx. 5 to 24 hours. After sedimentation, the glycerine phase is removed. Then the liquid phase is again compounded with methanol and potassium, or a sodium alkaline solution, if necessary, and the process of mixing and removal is repeated. The transesterified liquid is then neutralized with phosphoric acid, citric acid or other acids, whereby soap and the potassium or sodium salts of the acids are separated. In some cases, the transesterified liquid is rinsed with warm, deionized water, whereby the water absorbs the soap, alkaline solution, etc. This phase is also removed. Subsequently, all kinds of cleaning steps are possible. The process of stripping is also possible, whereby air flows in a counter flow to the ester in a scrubbing tower. The disadvantage of this process, which is based on a low-pressure transesterification process, is the long production time. Apart from the mixing process, long hold times are also required in the sedimentation phases.

U.S. Pat. No. 6,489,496 teaches the use of a high concentration of alcohol (8:1 mole ratio of alcohol to water) and recycle of a portion of the ester-alcohol product to create a single phase mixture to increase the rate of transesterification in the reaction zone. U.S. Pat. No. 4,695,411 teaches the use of hydrated ethyl alcohol in the presence of an acid catalyst as a means of creating a single phase in order to eliminate the two-phase barrier to the transesterification reaction. A reaction scheme that uses a co-solvent to create a miscible system to increase the reaction rate is also known.

Further, a method for the production of fatty acid methyl ester is known, wherein distillation is carried out after sedimentation of the glycerine solution in a separator, in order to purify and fractionate the methyl ester if required. Moreover, the reaction rate of the transesterification can be accelerated by increasing the temperature and with the help of alkaline or acid catalysts. The disadvantage of this method is the fact that the precipitation phase of the glycerine solution in the separator and even the possibility to accelerate the reaction time does not shorten the production time notably compared with the above state of the art.

AT-PS 398 777 contains a method for the cleaning of raw vegetable oil esters, whereby the vegetable oil ester is obtained by alkaline transesterification. The transesterification takes place with methanol in excess, with the addition of potassium hydroxide as a catalyst. The raw vegetable oil ester is treated with water vapor, whereby a glycerine phase is produced, which is removed. In this process, intensive mixing is required for transesterification, and the distilled alcohol can be recycled after recovery in a vacuum distillation column. Intensive mixing can also be achieved using centrifugal pumps, high shear mixers, or motionless mixers. See U.S. Pat. Nos. 6,015,440 and 6,174,501. The disadvantage of high intensity mixing is that the presence of soap materials, partially reacted triglycerides, or excess alcohol can lead to stable emulsions and very slow separation of the ester and glycerol phases. Additionally, U.S. Pat. Nos. 2,383,589 and 2,383,581 teach that the removal of any excess alcohol before separating the ester and glycerol phases results in a near-instantaneous separation.

U.S. Pat. No. 6,440,057 contains a process for the production of fatty acid methyl ester, whereby the fatty acids are subjected to high pressure, up to 200 bars, in a dynamic mixer whereby the emulsion may be subjected to ultrasound in order to increase the boundary interface. The disadvantage of such a method is the excessive pressures used in order to produce the optimum reaction, thus increasing production costs significantly. This method includes a number of filtration and centrifugation steps needed to separate the ester and glycerol phases because of very small, distributed glycerol bubbles and residual, excess alcohol creating a stable emulsion. Other methods based on high-pressure transesterification processes are also known. Transesterification takes place in an autoclave at very high temperatures, high pressure, and a large excess of alcohol with a relatively short reaction time. The disadvantage of such methods or equipment lies in the fact that an economic production of fatty acid methyl ester, for example for diesel fuel for diesel combustion engines, is absolutely impossible.

Furthermore, the transesterification process in two steps is also known. Thereby, the yield in terms of quantity and quality is certainly higher than with transesterification in one step, but again economic efficiency is not possible due to higher production costs. It is, therefore, desirable to provide a scalable apparatus and methodology for the production of fatty acid alkyl ester (i.e., biodiesel fuel) capable of speeding the transesterification process at only slightly elevated temperature and pressure.

### SUMMARY OF THE INVENTION

The present invention recognizes and addresses various of the foregoing limitations and drawbacks, and others, concerning the transesterification process for the production of fatty acid alkyl ester. Therefore, the present invention is directed to a method and apparatus for the production of fatty acid alkyl ester, in particular as fuel for diesel combustion engines.

It is, therefor, a principle object of the subject invention to provide a method for the production of fatty acid alkyl ester. More particularly, it is an object of the present invention to provide a method of producing diesel fuel. In such context, it is still a more particular object of the present invention to provide a methodology for the production of diesel fuel from vegetable oils or animal fats that does not require additional processing but that meets the ASTM specifications for biodiesel.

Still further, it is a principle object of this invention to provide a methodology for the efficient and economically viable production of fatty acid alkyl ester. In such context, it is an object of the present invention to provide such a methodology that is easily scalable from the laboratory to an industrial manufacturing facility with a capacity of one million gallons annual production to a facility with a production of more than 100 million gallons annual production.

Yet another principle object of the present invention is to provide an apparatus capable of performing such methodology. More particularly, it is an object of the present invention to provide an apparatus capable of speeding the transesterification reaction through the use of sonochemistry. In such context, it is a principle object of the present invention to accelerate such transesterification reaction at slightly elevated temperature and pressure levels.

Additional objects and advantages of the invention are set forth in, or will be apparent to those of ordinary skill in the art from, the detailed description as follows. Also, it should be further appreciated that modifications and variations to the specifically illustrated and discussed features and materials hereof may be practiced in various embodiments and uses of this invention without departing from the spirit and scope thereof, by virtue of present reference thereto. Such variations may include, but are not limited to, substitutions of the equivalent means, features, and materials for those shown or discussed, and the functional or positional reversal of various parts, features, or the like.

Still further, it is to be understood that different embodiments, as well as different presently preferred embodiments, of this invention, may include various combinations or configurations of presently disclosed features, elements, or their equivalents (including combinations of features or configurations thereof not expressly shown in the figures or stated in the detailed description).

These and other features, aspects, and advantages of the present invention will become better understood with reference to the following descriptions and appended claims. The accompanying drawings, which are incorporated in and constitute a part of this specification, illustrate an embodiment of the invention and, together with the descriptions, serve to explain the principles of the invention.

In one exemplary embodiment, there may be provided a method and apparatus for the production of fatty acid alkyl ester. The method of the present invention is characterized by boundary surfaces of a normally immiscible mixture, which are enlarged by ultrasonic irradiation. The present invention enables the production of fuel such as biodiesel in ecologically optimal conditions of production while maintaining all the advantages thereof.

The present invention accelerates the transesterification reaction by enlarging the boundary surfaces with dynamic processes. Through the use of high power ultrasonic cavitation, the size of the drops in the liquid phases may become effectively reduced, so that much smaller drops are produced, resulting in a much larger surface-to-volume ratio. This allows for the completed chemical reaction to be reached faster. The completed chemical reaction state is generally reached within between about twelve (12) and about eighteen (18) seconds.

Still further, transesterification may take place at between generally about 1.0 and generally about 5.0 atmospheres of pressure within the ultrasonic reactor thus eliminating the need for expensive high-pressure pumps. Chemical activation via sonochemistry, reactions induced by ultrasonic sound waves, is provided through the energy derived from the collapse of cavitation bubbles generated within the ultrasonic reactor. Such energy is derived when the cavitation bubbles collapse rapidly and violently and, in so doing, generate apparent temperatures of many thousands of degrees Kelvin and apparent pressures of several thousand atmospheres within the individual bubbles. Thus, the bubbles that are formed serve as individual microreactors operating at high temperature and high pressure while the liquid region immediately adjacent to the bubbles is at temperatures ranging generally from about 70° C. to generally about 80° C. and at between generally about 1.0 and generally about 5.0 atmospheres of pressure. Operating the present invention in excess of 70° C. at 1 atmosphere of pressure may promote the evaporation of the catalyst solution thus further achieving a complete chemical reaction at an accelerated rate, and using a previously unexpected low level of catalyst. Catalyst loadings of between about 0.2% to about 0.39% of catalyst per weight of fat or oil may be sufficient, compared with loadings of 0.4 to 1.18% of catalyst per weight of fat or oil in the presently known conventional processes. Further, excess alcohol loadings of generally from about 0.0% to generally about 2.4% above stoichiometric requirements per weight of fat or oil may be sufficient, compared with excess alcohol loadings of 50% to 200% of stoichiometric requirements per weight of fat or oil in the presently known conventional processes.

Additionally, transesterification may take place at frequencies of between about 20 kHz and about 50 kHz within the ultrasonic reactor. Still further, transestetification may take place at power densities of between about 18 Ws/ml and generally about 65 Ws/ml.

In accordance with one aspect of the present invention, transesterification takes place using fatty acids of vegetable oils or animal fats with an alkaline solution dissolved in near stoichiometric levels of alcohol to form a mixture where the alkyl esters are easily separated from the glycerine and soaps formed during the chemical reaction. Phase separation of the emulsion is achieved by natural gravitational separation. This gravitational separation process allows for the separation of the individual phases of the mixture. This allows the sedimentation step of the prior art to no longer be performed mechanically, for example, by filtration units. Instead the natural gravitational process of the present invention may occur over a significantly quicker time. Phase separation has been observed to take less than about sixty (60) seconds. This is the result of the absence of residual alcohol, which may act as a phase stabilizer between the ester and the glycerol phase. In such systems where residual alcohol is essentially absent, the catalyst is partitioned almost completely into the glycerol phase.

In accordance with another aspect of the present invention, the fatty acid alkyl ester may be cleaned in a wash solution and mechanically separated via centrifugal force. Due to the completeness of the transesterification reaction, minimal washing is required for removal of the catalyst, glycerol and soaps. The resultant product is a pure biodiesel fuel meeting all of the standards provided for by ASTM 6751-02, the current standard for biodiesel.

The apparatus envisioned for the implementation of the present invention may be characterized by the fact that it includes at least one container for the fats, and at least one tank each for the concentrated alkaline solution and the alcohol. Such containers are connected to the reaction section and there is a unit for separating the phases of the products downstream from the reaction section.

In accordance with an aspect of the present invention, the reaction section may comprise an ultrasonic flow through vessel. Such a design may provide for the creation of intense dynamic turbulence during the transesterification reaction with a simple device. Additionally, such a device is virtually maintenance free during operation and achieves the shortest possible reaction times without loss of quality or quantity.

In accordance with another aspect of the present invention, the unit for separating the phases of the emulsion is a natural gravity separatory unit. Such a device eliminates the need for the sedimentation phase of the prior art. Still further in accordance with a yet another aspect of the present invention, the device for washing the alkyl esters is a state of the art centrifuge that allows for both the washing and drying of the alkyl esters in one step to finally produce pure biodiesel.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 is a flow chart depicting the methodology of the present invention; and

FIG. 2 is an isometric view of the ultrasonic reactor of the present invention.

Repeat use of reference characters throughout the present specification and appended drawings is intended to represent the same or analogous features or elements of the invention.

### DETAILED DESCRIPTION OF THE PREFERRED EMBODIMENTS

Reference will now be made in detail to presently preferred embodiments of the invention, examples of which are fully represented in the accompanying drawings. Such examples are provided by way of an explanation of the invention, not limitation thereof. In fact, it will be apparent to those skilled in the art that various modifications and variations can be made in the present invention, without departing from the spirit and scope thereof For instance, features illustrated or described as part of one embodiment can be used on another embodiment to yield a still further embodiment. Still further, variations in selection of materials and/or characteristics may be practiced, to satisfy particular desired user criteria. Thus, it is intended that the present invention cover such modifications and variations as come within the scope of the present features and their equivalents.

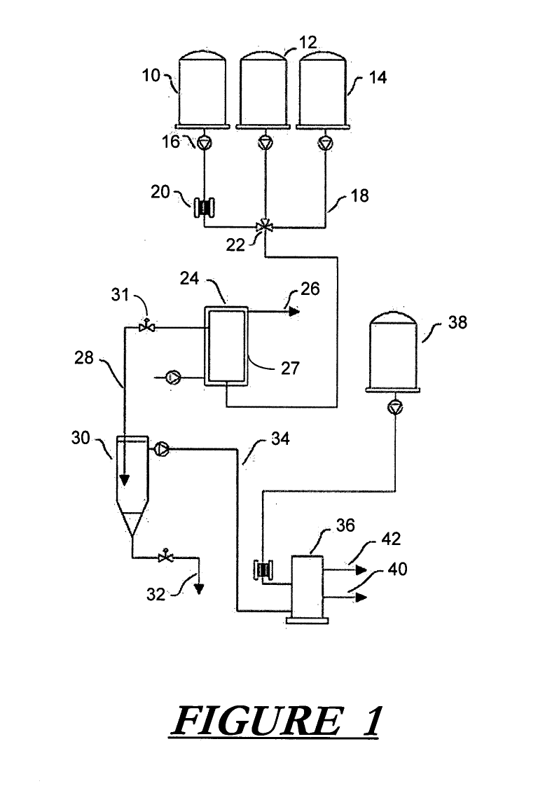

As disclosed above, the present invention is particularly concerned with an apparatus and method for the production of fatty acid alkyl ester. As seen in FIG. 1, a plurality of tanks for the chemical reactants involved in the present methodology is provided. A first tank 10 contains vegetable oil and/or animal fat. A second tank 12 is provided for a concentrated alkaline solution, particularly a sodium solution, and a third tank 14 is provided for the alcohol, particularly for methanol. Variable speed pumps 16 provide the fluid flow through connecting pipes 18.

A heat exchanger 20 provides the optimum animal fats and/or vegetable oil temperature prior to its combination with the alkaline and alcohol solution. The animal fat or vegetable oil is combined with the alkaline solution and the alcohol at a three-way connector 22 just prior to entering the ultrasonic reactor 24. A specific ultrasonic reactor temperature is maintained by cooling fluid 26, which is circulated by a pump through an outer cooling jacket 27 of the reactor 24. The emulsion is then subjected to ultrasonic irradiation within the ultrasonic reactor 24. Such exposure causes the emulsion to be transesterified.

The transesterified emulsion from the ultrasonic reactor 24 is connected via distribution pipes 28 to the separatory 30. The connecting pipe 28 is equipped with an additional control valve 31 to provide a constant pressure within the ultrasonic reactor 24. A glycerol solution is drained from the bottom of the separator 30 and sent to a repository through connecting pipe 32. Fatty acid methyl ester is pumped through connecting pipe 34 to a final wash centrifuge 36. The final wash solution from tank 38 is pumped through a heat exchanger 39 to provide optimum wash temperature prior to entering the final wash centrifuge 36. Spent wash fluid is removed from the final wash centrifuge 36 via port 40. To complete the final wash process pure biodiesel fuel is collected into storage vessels (not shown) through port 42 on the centrifuge 36.

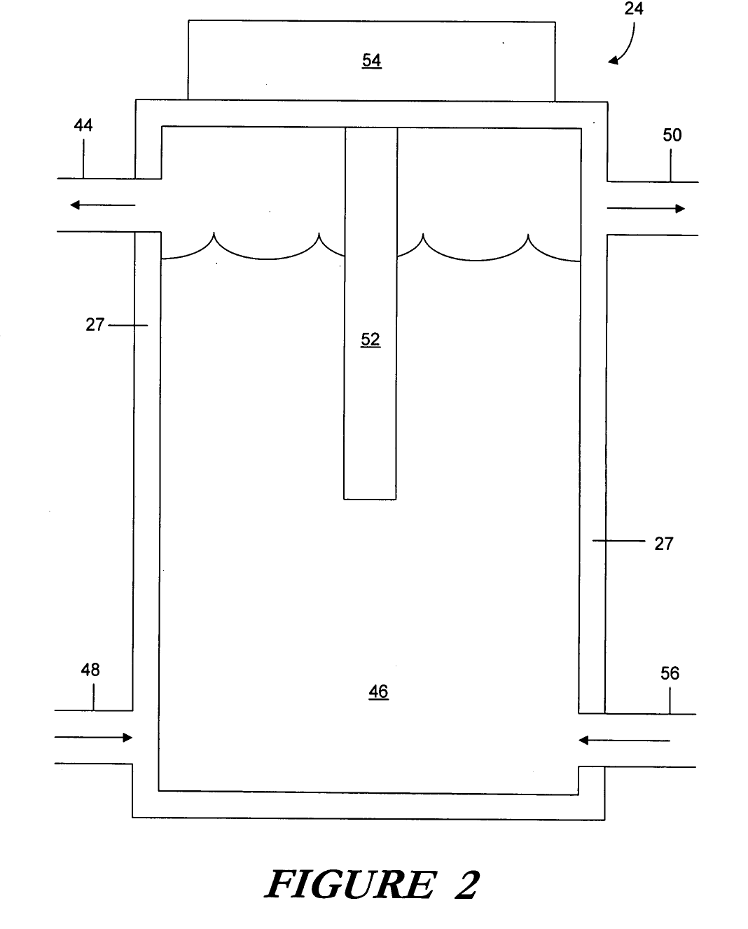

As best seen in FIG. 2, the ultrasonic flow-through reactor 24 comprises a continuous flow cell with a first inlet 44 for the emulsion 46 to be transesterified. As mentioned above, to maintain the reactor 24 at a specific temperature a cooling fluid 26 is circulated by a pump into inlet 48 through an outer cooling jacket 27 of the reactor 24 and back out through outlet 50. Within the flow-through reactor 24 is mounted a horn 52 connected to an ultrasonic generator 54 mounted so as to ensure a close proximity with the fluid 46 flow. Such configuration aids in generating an enlargement of the boundary surfaces. Boundary surface enlargement is achieved by reducing the droplet size of the fluid to be transesterified by ultrasonic cavitation. As transesterification is a boundary surface reaction, the enlarged surface areas correspondingly increase the transesterification reaction rate such that a chemical balance state is reached promptly.

To enhance the reaction time further, the process flow is carried out at specific temperatures, pressures, frequency, and energy density. Within the reactor 24 the process flow is carried out at specific temperatures of between generally about 70° C. to generally about 80° C. and at pressures of generally about 1.0 to generally about 5.0 atmospheres. Operation of the reactor 24 in excess of 70° C. at 1 atmosphere promotes the evaporation of the catalyst solution thus further achieving a complete chemical reaction at an enhanced rate, while requiring a significantly reduced level of catalyst and alcohol. Additionally, within the reactor 24 the process flow is subjected to ultrasonic irradiation at specific frequencies of between generally about 20 kHz and generally about 50 kHz. Finally, transesterification within the reactor 24 takes place at power densities of between generally about 18 Ws/ml and generally about 65 Ws/ml. Executing the transesterification process within these parameters and utilizing the present invention provides a chemical balanced state of the fatty acid methyl esters in times of less than eighteen (18) seconds.

Although a preferred embodiment of the invention has been described using specific terms and devices, such description is for illustrative purposes only. The words used are words of description rather than of limitation. It is to be understood that changes and variations may be made by those of ordinary skill in the art without departing from the spirit or the scope of the present invention, which is set forth in the following claims. In addition, it should be understood that aspects of various other embodiments may be interchanged both in whole or in part. Therefore, the spirit and scope of the appended claims should not be limited to the description of the preferred version contained herein.

What is claimed is:

1. A method for the production of fatty acid alkyl ester comprising the steps of:
a. providing an emulsion of naturally occurring fatty acids, an alkaline solution and an alcohol;
b. directly radiating said emulsion with ultrasonic energy to enhance the transesterification process;
c. separating said transesterified emulsion into a glycerol solution and fatty acid alkyl ester using a natural gravity separatory; and
d. washing and drying said fatty acid alkyl ester in a centrifuge to remove any remaining catalyst, alcohol, glycerol, soaps, and excess water.

2. The method of claim 1, wherein said naturally occurring fatty acids are animal fats.

3. The method of claim 1, wherein said naturally occurring fatty acids are vegetable oils.

4. The method of claim 1, wherein said alkaline solution is a concentrated form of one of the group comprising sodium hydroxide, potassium hydroxide, sodium methoxide, potassium methodoxide, or other strong mineral alkaline solutions.

5. The method of claim 1, wherein said alcohol is one of the group comprising methanol, ethanol, propanol, and other monoalkyl alcohols.

6. The method of claim 1, wherein said transesterification of said emulsion occurs in a reaction chamber, and wherein said reaction chamber comprises a horn.

7. The method of claim 6, wherein said horn ultrasonically irradiates said emulsion at frequencies between generally about 20 kHz to generally about 50 kHz.

8. The method of claim 6, wherein said horn operates at power densities of between generally about 18 Ws/ml and generally about 65 Ws/ml.

9. The method of claim 6, wherein said reaction chamber is maintained at a specific temperature of between generally about 70° C. and generally about 80° C. and an operating pressure of between generally about 1.0 and generally about 5.0 atmospheres.

10. The method of claim 1, wherein said alcohol loading of between generally about 0% to generally about 2.4% stoichiometric requirements per weight of said naturally occurring fatty acid is used.

11. A method for the production of an ASTM standard biodiesel fuel from an emulsion including fats, an alkaline solution and an alcohol, said method comprising the step of coupling ultrasonic energy to directly to said emulsion from a horn, wherein said ultrasonic energy is emitted to provide a density level of between about 18 Ws/ml and generally about 65 Ws/ml and at frequencies between about 20 kHz to generally about 50 kHz.

12. The method of Claim. 11, wherein said alkaline solution is a concentrated form of one of the group comprising sodium hydroxide, potassium hydroxide, sodium methoxide, potassium methodoxide, or other strong mineral alkaline solutions.

13. The method of claim 12, wherein said alcohol is one of the group comprising methanol, ethanol, propanol, and other monoalkyl alcohols.

14. The method of claim 13, wherein said alcohol is excess loaded in said emulsion between about 0% to generally about 2.4% of stoichiometric requirements per weight of said naturally occurring fatty acid.

15. An ASTM standard biodiesel fuel comprising a fatty acid alkyl ester separated from a transesterified emulsion of fats, alkaline solution and alcohol, said emulsion being transesterified in the presence of ultrasonic energy coupled directly therein at power densities of between about 18 Ws/ml to about 65 Ws/ml and frequencies of about 20 kHz to 80 kHz.

16. The biodiesel fuel of claim 15, wherein said alkaline solution is a concentrated form of one of the group comprising sodium hydroxide, potassium hydroxide, sodium methoxide, potassium methodoxide, or other strong mineral alkaline solutions.

17. The biodiesel fuel of claim 16, wherein said alcohol is one of the group comprising methanol, ethanol, propanol, and other monoalkyl alcohols.

18. The biodiesel fuel of claim 17, wherein said alcohol is excess loaded in said emulsion between about 0% to generally about 2.4% of stoichiometric requirements per weight of said naturally occurring fatty acid.
